(19) United States
(12) Patent Application Publication
(10) Pub. No.: US 2022/0111109 A1
(43) Pub. Date: Apr. 14, 2022

(54) AIR STERILIZER AND AIR STERILIZATION METHOD USING SAME

(71) Applicant: AweXome Ray, Inc., Anyang-si (KR)

(72) Inventors: Hong Sue CHOI, Anyang-si (KR); Jun Young CHOI, Anyang-si (KR); Se Hoon GIHM, Anyang-s (KR); Sung Hyun BAE, Anyang-si (KR); Nam Kyu LEE, Anyang-si (KR); Ki Hoon YOON, Anyang-si (KR); Chang Hyun KIM, Anyang-si (KR)

(21) Appl. No.: 17/450,611

(22) Filed: Oct. 12, 2021

(30) Foreign Application Priority Data
Oct. 12, 2020 (KR) 10-2020-0131160

(51) Int. Cl. A61L 9/20

(52) U.S. Cl. CPC A61L 9/20; A61L 2209/11; A61L 2209/12

(57) ABSTRACT

This application relates to an air sterilizer and an air sterilization method using same. In one aspect, the air sterilizer includes a housing including a flow path through which air moves. The air sterilizer may also include a first sterilization module configured to emit an electromagnetic wave to the air to sterilize the air, and a second sterilization module configured to remove a microorganism in the air using an electrostatic force. The first sterilization module may adjust a wavelength of the electromagnetic wave so that an intensity of the electromagnetic wave is determined based on a type of the microorganism, by controlling a tube voltage of the electromagnetic wave.

### CROSS-REFERENCE TO RELATED APPLICATION

This application claims priority to Korean Patent Application No. 10-2020-0131160 filed on Oct. 12, 2020. The entire contents of the application on which the priority is based are incorporated herein by reference.

### BACKGROUND

### Technical Field

The present disclosure relates to an air sterilizer and an air sterilization method using same.

### Description of Related Technology

With the progress of industrialization and the acceleration of urbanization and population density, a problem in pollution of air required for human breathing is getting serious day by day.

For example, due to the use of fossil fuels, pollutants harmful to humans are continuously released into the air, and the concentration of pathogenic microorganisms such as bacteria is also increasing in the air. Moreover, volatile organic compounds are included in the polluted air, and these volatile organic compounds include bacteria and viruses. These bacteria and viruses, and the volatile organic compounds and spores they produce, act as the cause of various diseases in humans.

### SUMMARY

The present disclosure provides an air sterilizer and an air sterilization method using an air sterilizer capable of effectively removing microorganisms (bacteria, virus, or the like) in air using electromagnetic waves and an electrostatic force.

However, aspects of the present disclosure are not limited to those mentioned herein, and other aspects that are not mentioned can be clearly understood by those of ordinary skill in the art to which the present disclosure belongs from the following description.

In accordance with a first aspect of the present application, there is provided an air sterilizer including: a housing including a flow path through which air moves; a first sterilization module configured to emit an electromagnetic wave to the air to sterilize the air; and a second sterilization module configured to remove a microorganism in the air using an electrostatic force.

The first sterilization module may adjust a wavelength of the electromagnetic wave so that an intensity of the electromagnetic wave is determined based on a type of the microorganism, by controlling a tube voltage of the electromagnetic wave.

The microorganism may include bacteria and viruses contained in the air, and the second sterilization module sterilizes the bacteria and reduces the viruses.

The first sterilization module may include: a charging case mounted to be attachable to and detachable from the housing; an electromagnetic wave tube provided in the charging case to emit the electromagnetic wave to the air; and a power supply configured to supply power to the electromagnetic wave tube.

The second sterilization module may include: a plurality of sterilization plates disposed to be spaced apart in a width direction of the flow path to remove the microorganism passing through spaces between the plurality of sterilization plates; and a sterilization electrode configured to apply power from an external power source to the sterilization plates to generate an electrostatic force on the sterilization plates.

The second sterilization module may include: an upper plate to which upper portions of the plurality of sterilization plates are coupled; a lower plate to which lower portions of the plurality of sterilization plates are coupled; a plurality of reinforcement pipes connecting both ends of the upper plate and both ends of the lower plate.

In accordance with a second aspect of the present disclosure, there is provided an air sterilization method including: emitting an electromagnetic wave to air introduced into the air sterilizer of claim 1 to sterilize the air; and removing a microorganism in the air using an electrostatic force.

The emitting the electromagnetic wave may include adjusting an wavelength of the electromagnetic wave so that an intensity of the electromagnetic wave is determined based on a type of the microorganism, by controlling a tube voltage of the electromagnetic wave.

The microorganism may include bacteria and viruses contained in the air, the emitting an electromagnetic wave includes ionizing the air through the electromagnetic wave, sterilizing the bacteria, and inactivating the viruses, and the removing the microorganism includes sterilizing the bacteria in the ionized air and reducing the viruses using the electrostatic force.

According to the present disclosure, after microorganisms (bacteria and viruses, or the like) contained in air are primarily removed through electromagnetic waves, subsequently, the microorganisms are secondarily removed using an electrostatic force, and thus, it is possible to effectively sterilize, inactivate, and reduce bacteria and viruses in the air.

### BRIEF DESCRIPTION OF THE DRAWINGS

FIG. 1 is a perspective view showing an air sterilizer according to an embodiment of the present disclosure.

FIG. 2 is a perspective view showing a lower module and an upper module separated in the air sterilizer according to the embodiment of the present disclosure.

FIG. 3 is a rear view showing a rear side of FIG. 1.

FIG. 4 is a perspective view of a state in which a first sterilization module and a second sterilization module are removed from the air sterilizer according to the embodiment of the present disclosure as viewed from above.

FIG. 5 is a rear view showing a state in which the first sterilization module and the second sterilization module are removed from the air sterilizer of FIG. 4 as viewed from the rear side.

FIG. 6 is a flowchart illustrating an air sterilization method according to another embodiment of the present disclosure.

### DETAILED DESCRIPTION

The desire of humans to breathe clean air is getting stronger, and an air sterilizer has been proposed to satisfy this desire. The air sterilizer can be largely classified into a filter sterilizer, an ion sterilizer, and a plasma sterilizer.

The filter sterilizer is a device that sucks air in a space to be purified, causes the air to passes through a filter, and adsorbs or decomposes contaminants in the air by the filter, and is most commonly used. However, in a case of the filter sterilizer, when the filter sterilizer is used for a long time, a maintenance action that requires periodic replacement of a new filter should be accompanied. Moreover, since characteristics of the filter are not perfect, the reliability of the air purification ability by this method is also not high.

The ion sterilizer increases the concentration of ions in the air to provide a purification or sterilization effect by negative ions. However, the ion sterilizer can expect a certain amount of purification or sterilization effect by the negative ions, but effects related to active removal of airborne bacteria may not be high.

The plasma sterilizer uses plasma cluster ion (PCI) technology to generate negative ions and positive ions in the air, and decomposes or inactivates harmful substances through a chemical reaction with harmful substances in the air to purify the air. However, the sterilization process by this method has not yet fully elucidated a chemical mechanism of PCI behavior, and since reliability of the sterilization effect is low, so far, the positive or negative effects from a microbiological point of view have not been fully identified.

Hereinafter, with reference to the accompanying drawings, the configuration and operation according to an embodiment of the present disclosure will be described in detail. The following description is one of several aspects of the disclosure that is claimable, and the description that follows may form a part of the detailed description of the present disclosure.

However, in describing the present disclosure, detailed descriptions of known configurations or functions may be omitted for clarity of the present disclosure.

Since the present disclosure may include various embodiments and various modifications, specific embodiments are illustrated in the drawings and described in the detailed description. However, this is not intended to limit the present disclosure to a specific embodiment, it should be understood to include all modifications, equivalents and substitutes included in the spirit and scope of the present disclosure.

Terms including ordinal numbers, such as first and second, may be used for describing various elements, but the corresponding elements are not limited by these terms. These terms are only used for the purpose of distinguishing one element from another element.

When an element is referred to as being ‘connected’ to, or ‘accessed’ to another element, it should be understood that the element may be directly connected to, or accessed to another element, but that other elements may exist in the middle.

The terms used in the present disclosure are only used for describing specific embodiments, and are not intended to limit the present disclosure. Singular expressions include plural expressions unless the context clearly indicates otherwise.

An exemplary embodiment of the present disclosure will be described in more detail with reference to the accompanying drawings.

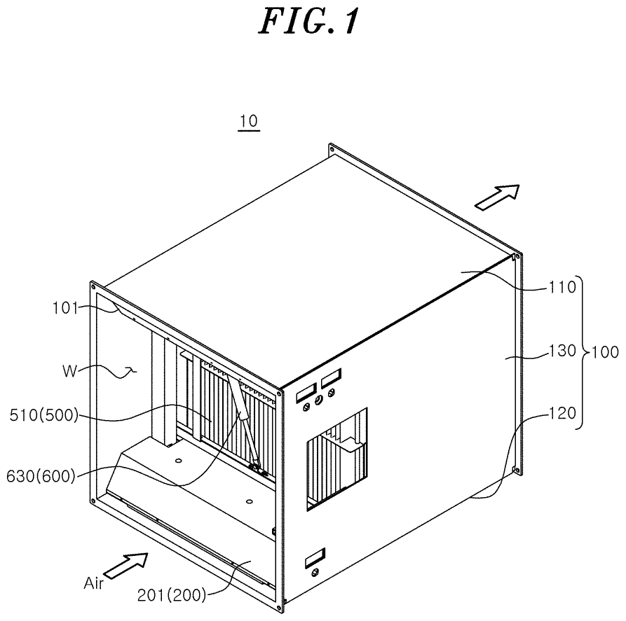

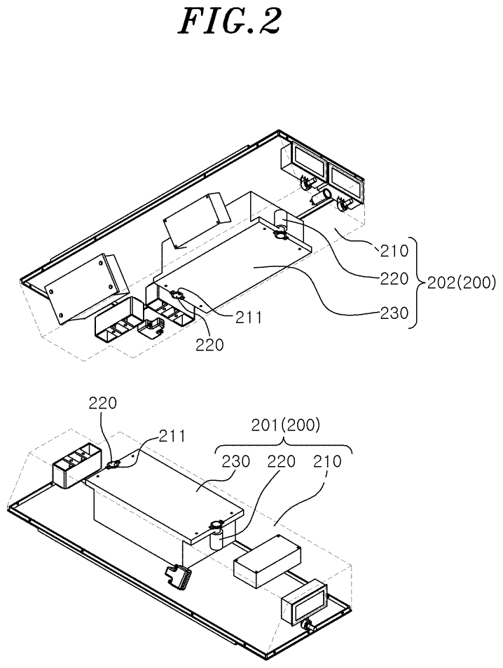

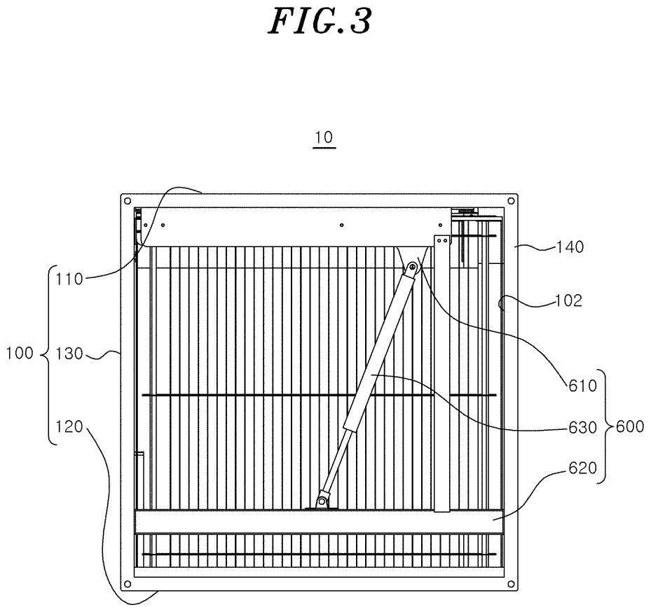

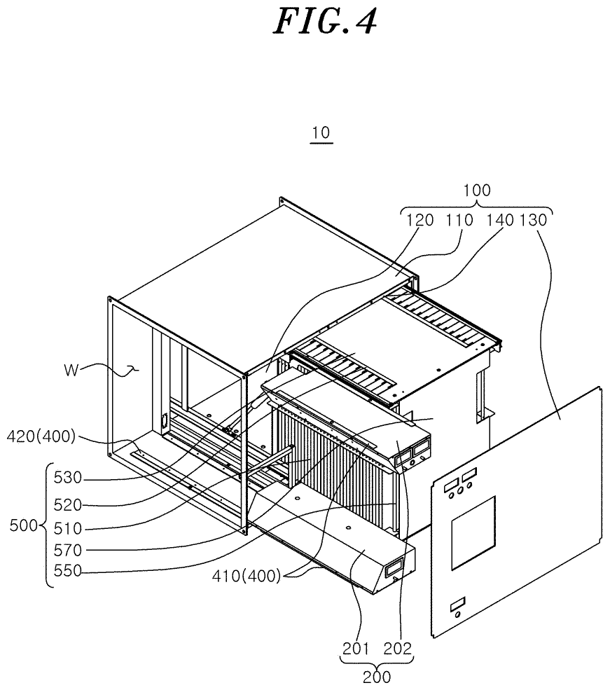

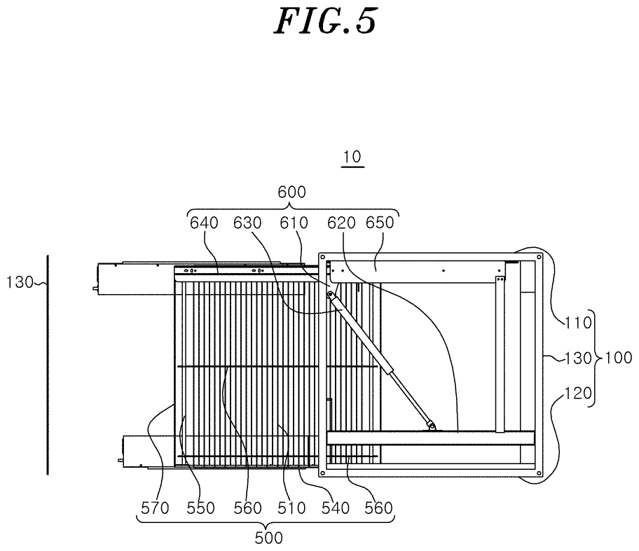

FIG. 1 is a perspective view showing an air sterilizer according to an embodiment of the present disclosure, FIG. 2 is a perspective view showing a lower module and an upper module separated in the air sterilizer according to the embodiment of the present disclosure, FIG. 3 is a rear view showing a rear side of FIG. 1, FIG. 4 is a perspective view of a state in which a first sterilization module and a second sterilization module are removed from the air sterilizer according to one embodiment of the present disclosure as viewed from above, and FIG. 5 is a rear view showing a state in which the first sterilization module and the second sterilization module are removed from the air sterilizer of FIG. 4 as viewed from the rear side.

Referring to FIGS. 1 to 5, the air sterilizer 10 according to the embodiment of the present disclosure may include a housing 100, a first sterilization module 200, and a second sterilization module 500.

Specifically, the housing 100 may constitute the overall appearance of the air sterilizer 10. An inlet 101 through which air is introduced may be provided at a front end portion of the housing 100, and an outlet 102 through which air is discharged may be provided at a rear end portion of the housing 100. A flow path W through which air moves may be provided inside the housing 100. The flow path W may serve as a passage through which air moves between the inlet 101 and the outlet 102.

A space in which the first sterilization module 200, a first attachment/detachment unit 400, the second sterilization module 500, and a second attachment/detachment unit 600 are installed may be provided in the housing 100. For example, a space in which the first sterilization module 200 and the first attachment/detachment unit 400 are installed may be provided on a side of the inlet 101 of the housing 100, and a space in which the second sterilization module 500 and the second attachment/detachment unit 600 are installed may be provided on a side of the outlet 102 of the housing 100.

The housing 100 may include an upper plate 110, a lower plate 120, a support frame 140, and side plates 130. The upper plate 110, the lower plate 120, and the side plates 130 are assembled to maintain a sealed state, and thus, it is possible to prevent electromagnetic waves generated by the first sterilization module 200 from leaking to the outside. The support frame 140 may be provided in the form of a square frame connecting the upper plate 110 and the lower plate 120 to each other. The side plate 130 may be bolted to the support frame 140.

A power socket for electrically connecting an external power source to the first sterilization module 200 and the second sterilization module 500 may be provided in at least one of the side plates 130 of the housing 100, and an instrument panel, a display, or the like for measuring and displaying various states of air in the housing 100 may be provided on the side plate 130. In particular, since the side plates 130 are assembled to the support frame 140 through a bolt, when replacement or upgrade of the first sterilization module 200 and the second sterilization module 500 is required, the side plates 130 can be easily removed from the support frame 140 after the bolt is removed.

The first sterilization module 200 may emit electromagnetic waves to the flow path W so that microorganisms in air are primarily removed. The microorganisms in the air may be removed by 40% or more by the first sterilization module 200.

The first sterilization module 200 includes a lower module 201 installed at an upper portion of the inlet 101 side of the housing 100 and an upper module 202 installed at an upper portion of the outlet 102 side of the housing 100. The lower module 201 and the upper module 202 may be disposed to face each other in a vertical direction in a state where the flow path W is interposed therebetween in the housing 100.

Each of the lower module 201 and the upper module 202 may include a charging case 210, an electromagnetic wave tube 220, and a power supply 230, respectively. The charging case 210 may be detachably mounted to the housing 100 through the first attachment/detachment unit 400. An electromagnetic wave hole 211 may be formed in the charge case 210. Since at least a portion of an electromagnetic wave emitting portion of the electromagnetic wave tube 220 is exposed to the electromagnetic wave hole 211, the electromagnetic waves may be emitted through the electromagnetic wave hole 211.

In addition, a voltmeter capable of monitoring a voltage of the power supply 230 and a voltage applied to the second sterilization module 500 may be installed in the charging case 210. In addition, an inclined surface for guiding the air introduced through the inlet 101 of the housing 100 to the second sterilization module 500 may be formed at a front end portion of the charging case 210. The inclined surface may be inclined upward from the inlet 101 of the housing 100 toward a center of the housing 100.

The electromagnetic wave tube 220 may be provided in the charging case 210 to emit electromagnetic waves toward the air moving through the flow path W. A position (that is, a position corresponding to the electromagnetic wave hole 211) at which the electromagnetic wave tube 220 is mounted on the charging case 210 may be determined (changed) based on ionization efficiency of air. Since ionization efficiency of the fine particles may be determined according to a volume to which the electromagnetic wave is emitted and a distance between fine particles, the electromagnetic wave tube 220 may be positioned at the position where the electromagnetic wave is emitted so that the ionization efficiency of the fine particles is maximized. According to the embodiment, the position to which the electromagnetic wave tube 220 is attached may be determined using artificial intelligence (AI).

The electromagnetic wave tube 220 emits electromagnetic waves to sterilize microorganisms in the air and ionize fine particles, thereby achieving charge balance in the air. Here, the electromagnetic wave may be provided in the form of ultraviolet rays, x-rays, and extreme ultraviolet rays (EUV) through control of electromagnetic wavelengths.

The electromagnetic wave tube 220 may emit an electromagnetic wave generated when electrons emitted from an emitter (for example, a carbon nanotube (CNT)-based emitter) collide with a target at a high speed in the air. The emitter may adjust the electromagnetic wavelength so that an intensity of the electromagnetic wave differs depending on a type of microorganism through change (regulation) of a tube voltage of the electromagnetic wave. For example, the emitter may adjust measured energy (eV). Here, the tube voltage may be understood as a maximum voltage given between an anode and a cathode of the electromagnetic wave tube 220.

In this way, when the tube voltage of the electromagnetic wave is adjusted, a wavelength, quality, dose, or the like of the electromagnetic wave generated from the electromagnetic wave tube 220 is changed, and thus, transmission power of the electromagnetic wave may be changed according to the type of microorganism. For example, when the wavelength of the electromagnetic wave generated from the electromagnetic wave tube 220 is adjusted to a wavelength of the ultraviolet light, nucleic acid of DNA of the microorganisms which is largely removed by the ultraviolet rays is destroyed or modified. Accordingly, the microorganisms may no longer be active, lose their ability to reproduce, and die. Of course, as factors affecting the sterilization effect, in addition to the wavelength of ultraviolet rays, there is an irradiation amount, humidity, a temperature, a wind speed, or the like. However, in general, the wavelength of ultraviolet rays may have the greatest effect.

Sterilization using x-rays may use a process of directly or indirectly ionizing the air introduced into the housing 100 to damage the DNA of microorganisms in the air. The sterilization action of the x-rays may be affected by environmental factors such as radiation sensitivity of air, temperature, oxygen conditions, and water activity.

For example, when an electromagnetic wave having a very short x-ray wavelength passes through a material, the electromagnetic wave ionizes atoms, groups, or molecules of the material to generate ions, and such an electromagnetic wave is called ionizing radiation. An irradiation technology of X-ray with the above characteristics enables cold-temperature sterilization without temperature increase. Specifically, these effects occur by damaging cellular components such as DNA and proteins of cells according to the direct and indirect actions of radiation. The direct action means that the energy of radiation is directly absorbed by organic molecules such as DNA of living things and causes damage such as structural changes in specific areas. Meanwhile, the indirect action means that molecules other than a target, such as water, which is the cytoplasmic solvent, absorb the energy of radiation to form an active substance such as a radical, and the active substance reacts with the target molecule to cause damage. Moreover, in terms of the biological effects of radiation, the effect of direct action corresponds to about 25% of the total effect, and the remaining 75% can be seen as the effect through indirect action. Particularly, hydroxyl radical (OH—) is known as the factor that has the greatest influence on the radiation susceptibility of organic organisms by occupying about 90% of the damage rate at the hydration interface of DNA molecules.

Factors affecting the sterilization of the x-rays include radiosensitivity and the surrounding environment.

In the case of radiosensitivity (sterilization object specificity), viruses, spore-forming bacteria, acellular bacteria (nutrient cells), yeast or mold, parasites, and insects have very different morphological differences. In particular, the response to the radiation is changed according to 1) cytoplasmic water content, 2) the size of the target chromosomal DNA, 3) the structures of repair enzymes and nucleases, 4) the diversity of genomic materials, or the like. Viruses in their simplest form do not have all the essential elements necessary for metabolism and are composed of nucleic acid genomes of DNA or RNA. In the case of bacterial spores, there is a relatively solidified “spore element” in the spore's core enzyme or DNA, as a resting structure with little or no free water in the cytoplasm, and the spore element is somewhat similar to bone tissue, and a film of the spore element is a multi-layer, impermeable protective film, which blocks radicals and toxins from the external environment generated by radiation, thus exhibiting high radiation resistance. Meanwhile, feeder cells have a genome that is 100 to 1000 times larger than that of viruses, and unlike spores, the genome is floating in the cytoplasm with a water content of 70 to 80%. Accordingly, the high-water content of feeder cells facilitates the formation of radicals in the cytoplasm, maximizing the effect on radiation. According to these characteristics, the feeder cells have more than 20 times higher radiation sensitivity than spores. Eukaryotic yeast or fungi have a larger genome than bacteria, and DNA thereof exists in a concentrated state of chromosomes surrounded by a nuclear membrane. Accordingly, the eukaryotic yeast or fungi may be easily destroyed by radiation, and more sensitive to radiation than prokaryotic cells.

In the case of the surrounding environment, the radiation sensitivity of microorganisms is changed according to a temperature, oxygen conditions, and water activity. Accordingly, the higher the temperature, the more oxygen, and the higher the water activity, the greater the effect of radiation on living things, and in this case, microorganisms can be sterilized with a relatively low radiation dose. For example, only heat treatment at 45° C., which is equal to or less than a lethal range of normal microorganisms, significantly increases the effect of irradiation on microorganisms. This is because a recovery action of microorganisms against DNA damage or the like does not work above a certain temperature. However, in the case of spores, due to their low moisture content, the effect of inhibiting the diffusion of additional radicals due to freezing is not large, and thus, the radiation resistance according to the freezing conditions is not significantly different. In addition, in general, the sterilization effect of radiation on microorganisms increases under aerobic conditions rather than anaerobic conditions. This is because a physiological metabolic process of microorganisms and a degree of radical generation are changed according to the presence or absence of oxygen. As a result of an experiment, it is known that in anaerobic conditions, the resistance of feeder cells increases 2 to 5 times compared to the aerobic system. In addition, the microorganisms are sensitive to radiation in an environment where the surrounding environment is high in moisture. This is because, in an environment with low moisture, the number of radicals generated by the irradiation decreases, and thus the degree of indirect action on DNA is also lowered. The strong resistance of bacterial spores to radiation is due to partial dehydration of the protoplasts. However, during germination of spores, the water activity in the protoplasm increases, and thus, the resistance to radiation is also lowered.

Meanwhile, the electromagnetic wave (x-ray) emitted from the electromagnetic wave tube 220 may be controlled so as not to be irradiated to the inlet 101 side of the housing 100. For example, by adjusting an angle at which the electromagnetic wave is irradiated from the electromagnetic wave tube 220, the electromagnetic wave may be adjusted to be irradiated toward the second sterilization module 500 side. As an example, the electromagnetic wave tube 220 may emit the electromagnetic waves in a range of greater than 0° and less than or equal to 90° to be irradiated toward the second sterilization module 500 side. The electromagnetic wave tube 220 may receive power from the power supply 230. The power supply 230 may receive power from an external power source to provide the power to the electromagnetic wave tube 220.

In the present embodiment, two electromagnetic wave tubes 220 are disposed to be spaced apart from the charging case 210, the numbers or positions of the electromagnetic wave tubes 220 are not limited thereto, and may vary as needed.

The first sterilization module 200 may be attached to or detached from the housing 100 through the first attachment/detachment unit 400. Here, the first attachment/detachment unit 400 may include a sliding bracket 410 extending to be bent from a lower portion of the first sterilization module 200 and a guide rail 420 installed in the housing 100 to correspond to the sliding bracket 410. The guide rail 420 may guide the movement of the sliding bracket 410 when the first sterilization module 200 is attached to or detached from the housing 100.

The second sterilization module 500 may use electrostatic force to secondarily remove microorganisms in the air. By the first sterilization module 200 and the second sterilization module 500, 99.9% or more of the microorganisms in the air can be removed.

The second sterilization module 500 may include a sterilization plate 510, a sterilization electrode 520, an upper plate 530, a lower plate 540, a reinforcement pipe 550, a gap reinforcement 560, and a side plate 570.

A plurality of sterilization plates 510 may be provided to provide an electrostatic force to the air ionized by the first sterilization module 200. The plurality of sterilization plates 510 may be disposed in the housing 100 in a state of being space apart at a predetermined interval in a width direction of the flow path W.

The sterilization electrode 520 may generate an electrostatic force in the sterilization plate 510 by applying a voltage (current) applied from an external power source to the plurality of sterilization plates 510. When the ionized air is moved between the plurality of sterilization plates 510, the microorganisms in the air converge on the plurality of sterilization plates 510 by the electrostatic force of the sterilization plate 510, and thus, bacteria in the air are sterilized and viruses are reduced. Air from which microorganisms are removed may be discharged to the outside of the second sterilization module 500 through the outlet 102 of the housing 100, that is, to the outside of the housing 100.

The upper plate 530 may be positioned at an upper portion of the second sterilization module 500, and may be connected to the lower plate 540 through the reinforcement pipe 550. Upper portions of the plurality of sterilization plates 510 may be coupled to the upper plate 530. At least a portion of the upper portions of the plurality of sterilization plates 510 may pass through the upper plate 530. The sterilization electrode 520 may be electrically connected to a portion of the upper portions of the sterilization plates 510 protruding through the upper plate 530.

The lower plate 540 may be located at a lower portion of the second sterilization module 500, and may be connected to the upper plate 530 through the reinforcement pipe 550. Lower portions of the plurality of sterilization plates 510 may be coupled to the lower plate 540. At least a portion of the lower portions of the plurality of sterilization plates 510 may pass through the lower plate 540. The sterilization electrode 520 may be electrically connected to a portion of the lower portions of the sterilization plates 510 protruding through the lower plate 540.

The plurality of reinforcement pipes 550 may be provided for the connection between the upper plate 530 and the lower plate 540. For example, upper ends of the plurality of reinforcement pipes 550 may be connected to both ends of the upper plate 530, and lower ends of the plurality of reinforcement pipes 550 may be connected to both ends of the lower plate 540.

A plurality of gap reinforcements 560 may be provided to fix both edges of the plurality of sterilization plates 510. Since the gap reinforcements 560 are fitted to both edges of the sterilization plate 510 and coupled thereto, the spacing between the plurality of sterilization plates 510 may be constantly maintained.

The second sterilization module 500 may be attached to or detached from the housing 100 by the second attachment/detachment unit 600. The second attachment/detachment unit 600 may include a support bracket 610, a housing bracket 620, an operating cylinder 630, a support rail 640, and a housing rail 650.

The support bracket 610 may be a mounting bracket installed on one side portion of an upper side of the second sterilization module 500. An upper end of the operating cylinder 630 may be hinged to the support bracket 610. The housing bracket 620 may be mounted on a lower inner portion of the housing 100. A lower end of the operating cylinder 630 may be hinged to the housing bracket 620.

The operating cylinder 630 may be connected between the support bracket 610 and the housing bracket 620. The operating cylinder 630 may provide a moving force for moving the second sterilization module 500 into and out of the housing 100 when the second sterilization module 500 is attached to or detached from the housing 100. For example, the operating cylinder 630 may be a gas (pneumatic) cylinder that provides a moving force to the second sterilization module 500 using gas pressure (pneumatic).

For example, the upper end of the operating cylinder 630 may be hinged to one end of the support bracket 610, and the lower end of the operating cylinder 630 may be hinged to a center portion of the housing bracket 620. Accordingly, when the first sterilization module 200 is attached to or detached from the housing 100, the operating cylinder 630 may be rotated while drawing an arc trajectory with the center of the housing bracket 620 as the center of rotation.

The support rail 640 may be mounted on the upper portion of the second sterilization module 500 so as to extend in a direction in which the second sterilization module 500 is attached or detached. One side of the support rail 640 may be fixedly installed on the upper portion of the second sterilization module 500, and the other side of the support rail 640 may be coupled to the housing rail 650 to be slidably moved along the housing rail.

The housing rail 650 may be installed at an upper portion of the housing 100 to correspond to the support rail 640. When the second sterilization module 500 is attached to or detached from the housing 100, the housing rail 650 supports the support rail 640 to be slidably moved, thereby guiding the support rail 640 in an attachable/detachable direction.

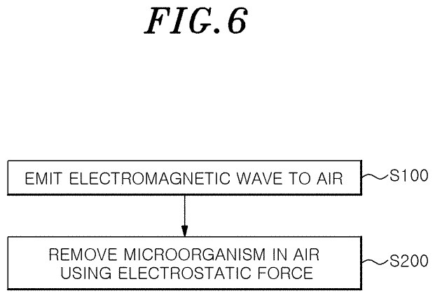

FIG. 6 is a flowchart illustrating an air sterilization method according to another embodiment of the present disclosure.

As shown in FIG. 6, the air sterilization method 20 according to another embodiment of the present disclosure may include a first sterilization step S100 and a second sterilization step S200.

In the first sterilization step S100, by emitting the electromagnetic waves to the air introduced through the inlet 101 of the air sterilizer 10, the microorganisms in the air are primarily sterilized. Here, the microorganism may include bacteria and viruses contained in the air. In this case, the air introduced into the inlet 101 of the housing 100 may move along the flow path W in the housing 100 and be discharged to the outlet 102 of the housing 100.

In the first sterilization step S100, the air may be ionized through the electromagnetic waves to primarily sterilize bacteria in the air, and inactivate viruses in the air. Through the first sterilization step S100, more than 40% of bacteria and viruses in the air can be removed.

In particular, in the first sterilization step S100, it is possible to emit the electromagnetic waves having different intensities of the electromagnetic waves according to the types of the microorganisms. Since the intensity of the electromagnetic wave that is fatal to the microorganisms is different depending on the types of the microorganisms, when the electromagnetic wave is emitted with the intensity of the electromagnetic wave that is most fatal to the microorganism, the microorganisms in the air can be effectively removed.

In the second sterilization step S200, the microorganisms in the air are secondarily removed using an electrostatic force. In the second sterilization step S200, it is possible to use the electrostatic force to secondarily sterilize the bacteria in the air ionized through the first sterilization step 200 and to reduce viruses.

As a result of the experiment, when the microorganisms in the air go through the first sterilization step S100 and the second sterilization step S200 in the air sterilizer 10, it was confirmed that 99.9% or more of the microorganisms in the air were removed.

The test results for a technical specificity of particle removal efficiency through x-ray and electrical attraction will be explained with a first comparative example to a third comparative example.

In the first comparative example, air is sterilized by emitting only x-rays within the housing 100 in a state where the output of the second sterilization module 500 was turned off. As a result, it was confirmed that colon bacillus (diameter of colon bacillus (d)=2.0 μm, concentration of colon bacillus (u)=100 lpm) was removed with the efficiency of approximately 40% while there was a difference in the sterilization rate according to an inflow concentration of colon bacillus (confirmed in test report).

In addition, in the second comparative example where x-rays were emitted to the air after the second sterilization module 500, only electrical attraction is applied, and a needle-type corona charger was used to increase electrical mobility of introduced aerosol particles, when a flow rate was increased (for example, from 1.2 lpm to 12.5 lpm), it was confirmed that the virus removal efficiency decreased at the virus 0.05 wt % condition. Moreover, when the flow rate was 100 lpm, it was confirmed that the removal efficiency was also relatively poor. In addition, when d=2.0 μm, the virus removal efficiency was expected to be less than 40%. Further, when only x-rays were emitted to the air, although there was a difference in the sterilization rate according to the inflow concentration of colon bacillus, the efficiency was approximately 40% (confirmed in test report). After removal of the microorganisms through the electrical attraction, when the microorganisms were removed through the x-ray irradiation, the expected value was expected to be less than 64%.

In addition, in the third comparative example where the x-ray and the electrical attraction of the second sterilization module 500 were simultaneously applied, it was confirmed that colon bacillus (d=2.0 μm, u=100 lpm) was removed with the efficiency of 99.9% (confirmed in test report).

As described above, compared to the removal of the microorganisms using only x-rays or the removal through the x-ray irradiation after removal of the microorganisms through the electrical attraction, in the removal of the microorganisms (particles) through the electrical attraction after the x-ray irradiation, when the x-ray and the electrical attraction of the second sterilization module 500 acted simultaneously, it was confirmed that the numerical improvement followed in the removal of colon bacillus.

Meanwhile, through test results 1 to 4 indicating the sterilization of the microorganisms below, it was confirmed that, in addition to colon bacillus, various types of microorganisms (P. aeruginosa, pneumococcus, MRSA, or the like) showed a 99.9% reduction rate before and after operation of the present disclosure.

(Test Result 1)

| | | | Test result | | | |
|---|---|---|---|---|---|---|
| | | | Concentration | | | |
| | | | before | Concentration | | |
| | | Test | operation | after operation | Reduction | Test |
| Test item | | method | (CFU/m²) | (CFU/m²) | ratio (%) | environment |
| Floating | AXR- | Presented | 2.2 × 10⁴ | <10 | 99.9 | (23.0 ± 0.2)° C. |
| microorganism | ESP- | by client | | | | (50.5 ± 2.0) % |
| reduction test | 003 | | | | | R.H. |
| (E. coli) | | | | | | |

(Test Result 2)

| | | | Test result | | | |
|---|---|---|---|---|---|---|
| | | | Concentration | Concentration | | |
| | | Test | before operation | after operation | Reduction | Test |
| Test item | | method | (CFU/m²) | (CFU/m²) | ratio (%) | environment |
| Floating | AXR- | Presented by | 2.1 × 10⁴ | <10 | 99.9 | (23.0 ± 0.2)° C. |
| microorganism | ESP- | client | | | | (50.5 ± 2.0) % |
| reduction test | 003 | | | | | R.H. |
| (P. aeruginosa) | | | | | | |

(Test Result 3)

| | | | Test result | | | |
|---|---|---|---|---|---|---|
| | | | Concentration | Concentration | | |
| | | Test | before operation | after operation | Reduction | Test |
| Test item | | method | (CFU/m²) | (CFU/m²) | ratio (%) | environment |
| Floating | AXR- | Presented | 1.8 × 10⁴ | <10 | 99.9 | (23.0 ± 0.2)° C. |
| microorganism | ESP- | by client | | | | (50.5 ± 2.0) % |
| reduction test | 003 | | | | | R.H. |
| (pneumococcus) | | | | | | |

(Test Result 4)

| | | | Test result | | | |
|---|---|---|---|---|---|---|
| | | | Concentration | | | |
| | | | before | Concentration | | |
| | | Test | operation | after operation | Reduction | Test |
| Test item | | method | (CFU/m²) | (CFU/m²) | ratio (%) | environment |
| Floating | AXR- | Presented by | 1.8 × 10⁴ | <10 | 99.9 | (23.0 ± 0.2)° C. |
| microorganism | ESP- | client | | | | (50.5 ± 2.0) % |
| reduction test | 003 | | | | | R.H. |
| (MRSA) | | | | | | |

In addition, through a test result 5 indicating the virus reduction below, it could be confirmed that floating virus (Phi-X174) showed a 74.5% reduction rate through the present disclosure.

(Test Result 5)

1. Test Result

result: floating virus reduction ratio: 74.5%

product name: air sterilization and purifying device

2. Test Item

(1) manufacturing cooperation: aweXome Ray Co. Ltd.

(2) model: AXR-ESP-003

3. Test Method and Condition

| Test method | Virus | Temperature | Humidity | Test chamber | Test time |
|---|---|---|---|---|---|
| KOUVA | Phi-X174 | (23 ± 2)° C. | (50 ± 5) % | 60 m³ | 30 minutes |
| AS 02: 2019 | (ATCC 13706-B1) | | R.H. | | |

Meanwhile, in the present disclosure, through field emission of carbon nanotubes, not only can an amount of electron emission be controlled through an electric field, but also wavelengths of electromagnetic waves can be controlled. In addition, as shown in Table 1 below, it can be confirmed that the carbon nanotube of the present disclosure exhibits an exceptionally good effect when used.

TABLE 1

| | AXRtube_10 kV | AXRtube_9 kV | AXRtube_8 kV |
|---|---|---|---|
| | applied | applied | applied |
| keV | (relative strength) | (relative strength) | (relative strength) |
| 5.0107 | 74129 | 59988 | 1479 |
| 5.0478 | 73773 | 59142 | 1375 |
| 5.0849 | 74232 | 59091 | 1361 |
| 5.122 | 74634 | 58470 | 1280 |
| 5.1591 | 74759 | 57683 | 1133 |
| 5.1962 | 75400 | 57251 | 1017 |
| 5.2333 | 75110 | 55944 | 941 |
| 5.2704 | 75200 | 54792 | 861 |
| 5.3075 | 75610 | 53601 | 730 |
| 5.3446 | 75219 | 52192 | 666 |
| 5.3817 | 74821 | 50936 | 579 |
| 5.4188 | 74580 | 49660 | 569 |
| 5.4559 | 74852 | 48046 | 476 |
| 5.493 | 74710 | 46429 | 402 |
| 5.5301 | 73827 | 44649 | 345 |

On the other hand, in the case of the existing x-ray light source technology using a filament, x-rays are generated by heating the filament to a temperature of several thousand degrees Celsius or more and causing emitted hot electrons to collide with a tungsten target. Then, in the filament of a certain specification, the hot electrons are emitted when energy more than critical energy (a certain temperature) is supplied. However, as shown in Table 2 below, when energy of the specific specification (for example, 5 kV) or more is applied, the filament is cut off. Accordingly, when the energy of the specific specification or more is applied, thermionic emission is not performed properly. Therefore, when application specifications are changed according to the usage environment, an inconvenience of replacing the tube itself may occur.

TABLE 2

| | 15 kV | 10 kV | 8 kV |
|---|---|---|---|
| | existing tube | existing tube | existing tube |
| keV | (relative strength) | (relative strength) | (relative strength] |
| 7.6448 | 17958 | 2846 | 0 |
| 7.6819 | 19253 | 3050 | 0 |
| 7.719 | 20026 | 3190 | 0 |
| 7.7561 | 20992 | 3337 | 0 |
| 7.7932 | 22024 | 3554 | 0 |
| 7.8303 | 22980 | 3729 | 0 |
| 7.8674 | 23720 | 3902 | 0 |
| 7.9045 | 24753 | 3902 | 0 |
| 7.9416 | 25880 | 4062 | 0 |
| 7.9787 | 26749 | 4049 | 0 |
| 8.0158 | 27449 | 3995 | 0 |
| 8.0529 | 28320 | 4033 | 0 |
| 8.09 | 29364 | 3991 | 0 |
| 8.1271 | 29770 | 3943 | 0 |

As an example, in Table 1, when 10 kV was applied to the carbon nanotubes of the present disclosure, it was confirmed that a peak was 75610, and in Table 2, when 10 kV was applied to the existing tube, it was confirmed that a peak was 4062. Accordingly, it can be seen that a 19 times difference occurs between the present disclosure and the existing tube.

In addition, as results of experiments to remove microorganisms in water, it was confirmed that although the emission energy of 7 kV is greater than the emission energy of 5 kV, the efficacy of the appropriate wavelength is better at 5 kV than at 7 kV. Although there is a difference in the environment of water rather than air, it has been confirmed that energy and removal efficiency are not proportional, and by changing the wavelength, it is possible to increase the sterilization efficacy through optimization for removing microorganisms (including viruses).

As described above, detailed descriptions of the present disclosure are made by the embodiments with reference to the accompanying drawings, but since the above-described embodiments have only been described with preferred examples of the present disclosure, the present disclosure is limited only to the above embodiments. The scope of the right of the present disclosure should be understood as the following claims and their equivalent concepts.

What is claimed is:

1. An air sterilizer comprising:
a housing including a flow path through which air moves;
a first sterilization module configured to emit an electromagnetic wave to the air to sterilize the air; and
a second sterilization module configured to remove a microorganism in the air using an electrostatic force.

2. The sterilizer of claim 1, wherein the first sterilization module is configured to adjust a wavelength of the electromagnetic wave so that an intensity of the electromagnetic wave is determined based on a type of the microorganism, by controlling a tube voltage of the electromagnetic wave.

3. The sterilizer of claim 1, wherein the microorganism includes bacteria and viruses contained in the air, and
wherein the second sterilization module is configured to sterilize the bacteria and reduces the viruses.

4. The sterilizer of claim 1, wherein the first sterilization module includes:
a charging case mounted to be attachable to and detachable from the housing;
an electromagnetic wave tube provided in the charging case to emit the electromagnetic wave to the air; and
a power supply configured to supply power to the electromagnetic wave tube.

5. The sterilizer of claim 1, wherein the second sterilization module includes:
a plurality of sterilization plates disposed to be spaced apart in a width direction of the flow path to remove the microorganism passing through spaces between the plurality of sterilization plates; and
a sterilization electrode configured to apply power from an external power source to the sterilization plates to generate an electrostatic force on the sterilization plates.

6. The sterilizer of claim 4, wherein the second sterilization module includes:
an upper plate to which upper portions of the plurality of sterilization plates are coupled;
a lower plate to which lower portions of the plurality of sterilization plates are coupled; and
a plurality of reinforcement pipes connecting both ends of the upper plate and both ends of the lower plate.

7. An air sterilization method comprising:
emitting an electromagnetic wave to air introduced into the air sterilizer of claim 1 to sterilize the air; and
removing a microorganism in the air using an electrostatic force.

8. The air sterilization method of claim 7, wherein the emitting includes adjusting a wavelength of the electromagnetic wave so that an intensity of the electromagnetic wave is determined based on a type of the microorganism, by controlling a tube voltage of the electromagnetic wave.

9. The air sterilization method of claim 7, wherein the microorganism include bacteria and viruses contained in the air,
wherein the emitting includes ionizing the air through the electromagnetic wave, sterilizing the bacteria, and inactivating the viruses, and
wherein the removing includes sterilizing the bacteria in the ionized air and reducing the viruses using the electrostatic force.
